(19) United States
(12) Patent Application Publication
(10) Pub. No.: US 2020/0365266 A1
(43) Pub. Date: Nov. 19, 2020

(54) SYSTEMS AND METHODS FOR PROVIDING POSTURE FEEDBACK AND HEALTH DATA BASED ON MOTION DATA, POSITION DATA, AND BIOMETRIC DATA OF A SUBJECT

(71) Applicant: Duke University, Durham, NC (US)

(72) Inventors: Leighanne Jarvis, Durham, NC (US); Kevin Caves, Durham, NC (US)

(21) Appl. No.: 16/816,163

(22) Filed: Mar. 11, 2020

Related U.S. Application Data
(60) Provisional application No. 62/817,725, filed on Mar. 13, 2019.

(51) Int. Cl. G16H 40/67; G16H 50/30; G16H 50/20; G16H 15/00; G16H 20/30; G06N 20/00; A61B 5/11; A61B 5/00; A61B 5/0205; H04W 4/38

(52) U.S. Cl. CPC G16H 40/67; A61B 5/02438; G16H 50/20; G16H 15/00; G16H 20/30; G06N 20/00; A61B 5/1116; A61B 5/4815; A61B 5/681; A61B 5/6898; A61B 5/0205; A61B 5/486; A61B 5/1118; A61B 5/7267; A61B 5/742; A61B 5/0002; H04W 4/38; G16H 50/30

(57) ABSTRACT

Systems and methods for providing posture feedback and health data based on motion data, position data, and biometric data of a subject. According to an aspect, a system includes one or more sensors configured to capture motion data, position data, and/or biometric data associated with a subject. The system also includes a monitor configured to determine at least one indicator of one of posture and activity of the subject based on the captured motion data, position data, and/or biometric data. The monitor is also configured to analyze the at least one indicator to determine health data of the subject. Further, the monitor is configured to present the health data.

### CROSS REFERENCE TO RELATED APPLICATION

This application claims priority to U.S. Provisional Patent Application No. 62/817,725, filed Mar. 13, 2019, and titled SYSTEMS AND METHODS FOR TRACKING BODY POSITION, the content of which is incorporated herein by reference in its entirety.

### TECHNICAL FIELD

The presently disclosed subject matter relates generally to healthcare devices. Particularly, the presently disclosed subject matter relates to systems and methods for providing posture feedback and health data based on motion data, position data, and biometric data of a subject.

### BACKGROUND

While many factors affect physical resilience, health and fitness are contributing factors. Accelerometer-based activity monitors have been used with increasing frequency in both research and clinical environments. Several studies have used accelerometry in inpatient and outpatient settings to gather activity data, but many more rely on patient reported outcomes or reports from staff or family members. More than one-third of hospitalized elders are discharged with a major new functional disability in performing basic activities of daily living. A key barrier to improving hospital mobility has been lack of a reliable and clinically meaningful way to measure mobility in the inpatient setting. Therefore, there is a need for improved systems to track and report patient mobility.

### BRIEF DESCRIPTION OF THE DRAWINGS

Having thus described the presently disclosed subject matter in general terms, reference will now be made to the accompanying Drawings, which are not necessarily drawn to scale, and wherein:

FIG. 1 is a diagram of a system for providing health information based on motion and/or position data of a subject in accordance with embodiments of the present disclosure;

FIG. 2 is a flow diagram of a method for providing health information based on motion and/or position data of a subject in accordance with embodiments of the present disclosure;

FIG. 3 is a flow diagram of a method for monitoring and/or tracking a position and/or posture of a subject in accordance with embodiments of the present disclosure;

FIG. 4 is a flow diagram of a method for monitoring and/or tracking a position and/or posture of a subject in accordance with embodiments of the present disclosure;

FIG. 5 is a flow diagram of a method for monitoring and/or tracking a position and/or posture of a subject in accordance with embodiments of the present disclosure;

FIG. 6 is a diagram depicting application of an algorithm to training data and test data to generate results in accordance with embodiments of the present disclosure;

FIG. 7A is a diagram showing the progression of states of the patient beginning with the state of laying to the state of walking;

FIG. 7B shows an example screen display of the smartphone in accordance with embodiments of the present disclosure;

FIG. 7C is a graph showing results of initial trails; and

FIG. 8 is a confusion matrix of data for a hospitalized adult.

### SUMMARY

The presently disclosed subject matter relates to systems and methods for providing posture feedback based on motion data. According to an aspect, a system includes one or more sensors configured to capture motion data, position data, and/or biometric data associated with a subject. The system also includes a monitor configured to determine at least one indicator of one of posture and activity of the subject based on the captured motion data, position data, and/or biometric data. The monitor is also configured to analyze the at least one indicator to determine health data of the subject. Further, the monitor is configured to present the health data.

### DETAILED DESCRIPTION

The following detailed description is made with reference to the figures. Exemplary embodiments are described to illustrate the disclosure, not to limit its scope, which is defined by the claims. Those of ordinary skill in the art will recognize a number of equivalent variations in the description that follows.

Articles “a” and “an” are used herein to refer to one or to more than one (i.e. at least one) of the grammatical object of the article. By way of example, “an element” means at least one element and can include more than one element.

“About” is used to provide flexibility to a numerical endpoint by providing that a given value may be “slightly above” or “slightly below” the endpoint without affecting the desired result.

The use herein of the terms “including,” “comprising,” or “having,” and variations thereof is meant to encompass the elements listed thereafter and equivalents thereof as well as additional elements. Embodiments recited as “including,” “comprising,” or “having” certain elements are also contemplated as “consisting essentially of” and “consisting” of those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a range is stated as between 1%-50%, it is intended that values such as between 2%-40%, 10%-30%, or 1%-3%, etc. are expressly enumerated in this specification. These are only examples of what is specifically intended, and all possible combinations of numerical values between and including the lowest value and the highest value enumerated are to be considered to be expressly stated in this disclosure.

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical terms used herein have the same meaning as commonly understood by one of ordinary skill in the art to which this disclosure belongs.

The functional units described in this specification have been labeled as computing devices. A computing device may be implemented in programmable hardware devices such as processors, digital signal processors, central processing units, field programmable gate arrays, programmable array logic, programmable logic devices, cloud processing systems, or the like. The computing devices may also be implemented in software for execution by various types of processors. An identified device may include executable code and may, for instance, comprise one or more physical or logical blocks of computer instructions, which may, for instance, be organized as an object, procedure, function, or other construct. Nevertheless, the executable of an identified device need not be physically located together but may comprise disparate instructions stored in different locations which, when joined logically together, comprise the computing device and achieve the stated purpose of the computing device. In another example, a computing device may be a mobile computing device such as, for example, but not limited to, a smart phone, a cell phone, a pager, a personal digital assistant (PDA), a mobile computer with a smart phone client, an Internet of Thing (IoT) device, a microcontroller-based device, or the like. In another example, a computing device may be any type of wearable computer, such as a computer with a head-mounted display (HMD), or a smartwatch or some other wearable smart device. Some of the computer sensing may be part of the fabric of the clothes the user is wearing. A computing device can also include any type of conventional computer, for example, a laptop computer or a tablet computer. A typical mobile computing device is a wireless data access-enabled device (e.g., an iPHONE® smart phone, a BLACKBERRY® smart phone, a NEXUS ONE™ smart phone, an iPAD® device, smart watch, or the like) that is capable of sending and receiving data in a wireless manner using protocols like the Internet Protocol, or IP, and the wireless application protocol, or WAP. This allows users to access information via wireless devices, such as smart watches, smart phones, mobile phones, pagers, two-way radios, communicators, and the like. Wireless data access is supported by many wireless networks, including, but not limited to, Bluetooth, Near Field Communication, CDPD, CDMA, GSM, PDC, PHS, TDMA, FLEX, ReFLEX, iDEN, TETRA, DECT, DataTAC, Mobitex, EDGE and other 2G, 3G, 4G, 5G, and LTE technologies, WI-FI® communications technologies, and it operates with many handheld device operating systems, such as PalmOS, EPOC, Windows CE, FLEXOS, OS/9, JavaOS, iOS and Android. Typically, these devices use graphical displays and can access the Internet (or other communications network) on so-called mini- or micro-browsers, which are web browsers with small file sizes that can accommodate the reduced memory constraints of wireless networks. In a representative embodiment, the mobile device is a cellular telephone or smart phone or smart watch that operates over GPRS (General Packet Radio Services), which is a data technology for GSM networks or operates over Near Field Communication e.g. Bluetooth. In addition to a conventional voice communication, a given mobile device can communicate with another such device via many different types of message transfer techniques, including Bluetooth, Near Field Communication, SMS (short message service), enhanced SMS (EMS), multi-media message (MMS), email WAP, paging, or other known or later-developed wireless data formats. Although many of the examples provided herein are implemented on smart phones, the examples may similarly be implemented on any suitable computing device, such as a computer.

An executable code of a computing device may be a single instruction, or many instructions, and may even be distributed over several different code segments, among different applications, and across several memory devices. Similarly, operational data may be identified and illustrated herein within the computing device, and may be embodied in any suitable form and organized within any suitable type of data structure. The operational data may be collected as a single data set, or may be distributed over different locations including over different storage devices, and may exist, at least partially, as electronic signals on a system or network.

The described features, structures, or characteristics may be combined in any suitable manner in one or more embodiments. In the following description, numerous specific details are provided, to provide a thorough understanding of embodiments of the disclosed subject matter. One skilled in the relevant art will recognize, however, that the disclosed subject matter can be practiced without one or more of the specific details, or with other methods, components, materials, etc. In other instances, well-known structures, materials, or operations are not shown or described in detail to avoid obscuring aspects of the disclosed subject matter.

As used herein, the term “memory” is generally a storage device of a computing device. Examples include, but are not limited to, read-only memory (ROM) and random access memory (RAM).

The device or system for performing one or more operations on a memory of a computing device may be a software, hardware, firmware, or combination of these. The device or the system is further intended to include or otherwise cover all software or computer programs capable of performing the various heretofore-disclosed determinations, calculations, or the like for the disclosed purposes. For example, exemplary embodiments are intended to cover all software or computer programs capable of enabling processors to implement the disclosed processes. Exemplary embodiments are also intended to cover any and all currently known, related art or later developed non-transitory recording or storage mediums (such as a CD-ROM, DVD-ROM, hard drive, RAM, ROM, floppy disc, magnetic tape cassette, etc.) that record or store such software or computer programs. Exemplary embodiments are further intended to cover such software, computer programs, systems and/or processes provided through any other currently known, related art, or later developed medium (such as transitory mediums, carrier waves, etc.), usable for implementing the exemplary operations disclosed below.

In accordance with the exemplary embodiments, the disclosed computer programs can be executed in many exemplary ways, such as an application that is resident in the memory of a device or as a hosted application that is being executed on a server and communicating with the device application or browser via a number of standard protocols, such as TCP/IP, HTTP, XML, SOAP, REST, JSON and other sufficient protocols. The disclosed computer programs can be written in exemplary programming languages that execute from memory on the device or from a hosted server, such as BASIC, COBOL, C, C++, Java, Pascal, or scripting languages such as JavaScript, Python, Ruby, PHP, Perl, or other suitable programming languages.

As referred to herein, the terms “computing device” and “entities” should be broadly construed and should be understood to be interchangeable. They may include any type of computing device, for example, a server, a desktop computer, a laptop computer, a smart phone, a cell phone, a pager, a personal digital assistant (PDA, e.g., with GPRS NIC), a mobile computer with a smartphone client, or the like.

As referred to herein, a user interface is generally a system by which users interact with a computing device. A user interface can include an input for allowing users to manipulate a computing device, and can include an output for allowing the system to present information and/or data, indicate the effects of the user's manipulation, etc. An example of a user interface on a computing device (e.g., a mobile device) includes a graphical user interface (GUI) that allows users to interact with programs in more ways than typing. A GUI typically can offer display objects, and visual indicators, as opposed to text-based interfaces, typed command labels or text navigation to represent information and actions available to a user. For example, an interface can be a display window or display object, which is selectable by a user of a mobile device for interaction. A user interface can include an input for allowing users to manipulate a computing device, and can include an output for allowing the computing device to present information and/or data, indicate the effects of the user's manipulation, etc. An example of a user interface on a computing device includes a graphical user interface (GUI) that allows users to interact with programs or applications in more ways than typing. A GUI typically can offer display objects, and visual indicators, as opposed to text-based interfaces, typed command labels or text navigation to represent information and actions available to a user. For example, a user interface can be a display window or display object, which is selectable by a user of a computing device for interaction. The display object can be displayed on a display screen of a computing device and can be selected by and interacted with by a user using the user interface. In an example, the display of the computing device can be a touch screen, which can display the display icon. The user can depress the area of the display screen where the display icon is displayed for selecting the display icon. In another example, the user can use any other suitable user interface of a computing device, such as a keypad, to select the display icon or display object. For example, the user can use a track ball or arrow keys for moving a cursor to highlight and select the display object.

The display object can be displayed on a display screen of a mobile device and can be selected by and interacted with by a user using the interface. In an example, the display of the mobile device can be a touch screen, which can display the display icon. The user can depress the area of the display screen at which the display icon is displayed for selecting the display icon. In another example, the user can use any other suitable interface of a mobile device, such as a keypad, to select the display icon or display object. For example, the user can use a track ball or times program instructions thereon for causing a processor to carry out aspects of the present disclosure.

As referred to herein, a computer network may be any group of computing systems, devices, or equipment that are linked together. Examples include, but are not limited to, local area networks (LANs) and wide area networks (WANs). A network may be categorized based on its design model, topology, or architecture. In an example, a network may be characterized as having a hierarchical internetworking model, which divides the network into three layers: access layer, distribution layer, and core layer. The access layer focuses on connecting client nodes, such as workstations to the network. The distribution layer manages routing, filtering, and quality-of-server (QoS) policies. The core layer can provide high-speed, highly-redundant forwarding services to move packets between distribution layer devices in different regions of the network. The core layer typically includes multiple routers and switches.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some embodiments, the patient comprises a human.

As used herein, the terms “position” and “posture” are used interchangeably and refer to the position of a subject's body in relation to the environment. Examples of positions/postures include, but are not limited to, sitting, standing, reclining, laying and walking.

As used herein, the term “health data” refers to any data or information indicative of a condition of a subject or patient. Health data can be used to indicate the current patient state or the progression of a subject between different states of mobility such as, but not limited to, laying, reclining, sitting, walking, jogging, running, or the like. Health data can be aggregated and used to display reports on activity in a period that can be used by staff to modify care planning. Additionally, health data can be used to provide feedback to the patient that can be used for tracking progress, reminders or goal settings. A user interface (e.g., a display) may be used for presenting health data to a subject. In examples, the user interface may be presented by text, audibly, graphically (e.g., graphs), and the like.

As used herein, the term “motion data” refers to any data indicative of movement or non-movement of a subject or patient. For example, motion data may indicate that there has been no change in the position of a subject. In another example, motion data may indicate a distance all or a portion of the subject has moved. The motion data may indicate a speed or velocity that all or a portion of the subject has moved. For example, the motion data may indicate that the subject has walked a distance in a time period or at a particular speed. Motion data may be acquired or captured by suitable electronic devices or components such as an accelerometer, gyroscope, or global positioning system (GPS).

As used herein, the term “position data” refers to any data indicative of a state of position of a subject or patient. For example, position data may indicate that the subject is upright, reclined, or laying down. Further, the position data may indicate that the position of a limb of a person relative to another limb such that the state (e.g., reclined or walking) can be determined or deduced. Position data may be acquired or captured by suitable electronic devices or components such as an accelerometer, gyroscope, or GPS receiver.

As used herein, the term “biometric data” refers to any data indicative of a health condition or state of a subject or patient. For example, biometric data can include heartrate, heart rate variability, temperature, blood pressure, galvanic skin response, sleep quality, etc. Multiple biometric data streams can be used to improve the system performance, for example, looking at heart rate in combination with accelerometry can help to distinguish periods of standing from walking in subjects who move extremely slowly.

The presently disclosed subject matter is directed to systems, methods, and devices that monitor and track a subject's or individual's body position between different phases of movement. For example, the movement may include, but is not limited to, laying, reclining, sitting, standing, walking, or the like. Systems and methods disclosed herein can be used in hospitals (e.g., inpatient settings) and with populations in other environments (e.g., at a residence) where movement or posture monitoring can be helpful. In a hospital setting for example, systems and methods disclosed herein may be used to monitor the position and/or posture of a patient. As an example, systems and methods disclosed herein may be utilized by subjects with conditions such as Parkinson's Disease, older adults, occupations that suffer from bad posture (e.g., surgeons, computer programmers, etc.). In a residence environment for example, systems and methods disclosed herein may be utilized with a senior or elderly subject with limited mobility or confined to a bed for adjusting posture and/or positioning to avoid bed sores. In some embodiments, systems and methods disclosed herein utilize an algorithm with automated data processing and high-performance machine learning (ML) techniques on data collected from an accelerometer or other motion sensing component worn by a subject to monitor postural changes and provide real time feedback for self-correction or intervention.

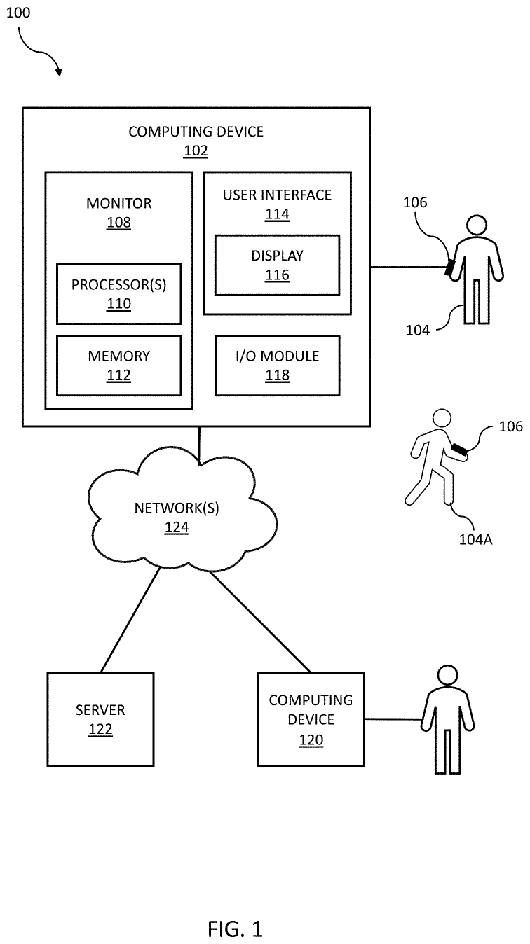

FIG. 1 illustrates a diagram of a system 100 for providing health information based on motion and/or position data of a subject in accordance with embodiments of the present disclosure. Referring to FIG. 1, the system 100 includes a computing device 102 that is carried by or otherwise associated with a subject 104 who is being monitored. The computing device 102 may be a smartphone, a tablet computer, a laptop computer, or the like. The system 100 may also include a sensor 106 configured to capture motion and/or position data associated with the subject 104. The sensor 106 and the computing device 102 may be communicatively connected via a wired connection or wireless connection (e.g., BLUETOOTH® wireless technology) for communication of captured motion and/or position data from the sensor 106 to the computing device 102. In an example, the sensor 106 may include an accelerometer configured to acquire motion data of the subject 104 and to communicate the acquired motion data to the computing device 102. In accordance with embodiments, the computing device 102 includes a monitor 108 configured to determine one or more indicators of posture and/or activity of the subject 104 based on the captured motion and/or position data, to analyze the indicator(s) to determine health data of the subject 104, and to present the determined feedback. It is noted that although only one sensor 106 is shown in the example of FIG. 1, there may be more than one sensor for use in capturing motion and/or position data of the subject 104.

The monitor 108 may include suitable hardware, software, firmware, or combinations thereof for implementing the functionality described herein. For example, the monitor 108 may include memory 110 and one or more processor(s) 112 configured to implement instructions for implementing the functionality described herein. In an example, the monitor 108 may be implemented as an application (or “app”) that resides on the computing device 102. The subject 104 or another person may interact with the monitor 108 via a user interface 114. For example, the user interface 114 may be a touchscreen display 116 for displaying health data and other information and for receiving commands or other input as disclosed herein. As an example, the subject 104 may view on the display data (e.g., extracted information, feedback information, etc.) that originate from or are triggered by the sensor. In addition, for example, the subject 104 may select part of the data for storage on the server 122 or other external storage device.

The sensor 106 may be any suitable device or component configured to capture motion and/or position data of a subject. For example, the sensor 106 may be an accelerometer, a gyroscope, a heart monitor, a beacon component, a position sensor (e.g., a GPS receiver), or the like. One or more of the sensors may be configured to communicate to the computing device 102 their captured data. Further, the sensor(s) may be configured to be attached to the subject's clothing, upper torso, and leg while capturing the motion and/or position data. In an example, the sensor 106 may be positioned such that, during its operation, accelerometer data relating to the posture of the subject 104 can be obtained or acquired. Suitable positions for placement of the sensor 106 include, but are not limited to, the shirt collar, front or back of the shirt, belt/waistline, glasses, and the like.

The computing device 102 may include an input/output (I/O) module 118 configured to communicatively interface with other computing devices. For example, the I/O module 118 may be configured to wirelessly communicate directly with the sensor 106 via a short range wireless connection (e.g., BLUETOOTH® communication). The I/O module 118 may also be configured to wirelessly (or via a wired connection) communicate with another computing device 120 or server 122 via one or more communications networks 124.

The sensor 106 can be configured to communicate with the computing device 102 for analyzing the captured motion and/or position data (e.g., accelerometer data) in accordance with embodiments of the present disclosure. As discussed in further detail, the computing device 102 can perform logic operations on one or more inputs of motion data, position data, and/or other data according to stored or accessible software instructions providing functionality as disclosed herein. In some embodiments, computing device 102 may present feedback to the subject 104, including information based on the analyzed motion and/or position data and the stored software instructions. In some embodiments, the computing device 102 may access memory (e.g., memory 112 or in the remote server 122) where executable instructions are stored or, in other embodiments.

In accordance with embodiments, a computing device in combination with one or more sensors, such as computing device 102 and sensor 106, are configured to analyze the data captured by the sensor(s) to determine one or more indicators of posture of the subject. Further, the computing device can determine feedback for presentation to the subject based on the indicator(s) of the posture of the subject. The computing device as provided herein may be configured to identify one or more posture indicators obtained from the accelerometer data, or other motion and/or position data. In some embodiments, the computing device generates a data log from some or all of the received accelerometer data, including data related to posture indicators. The posture indicator(s) may be associated with any position/posture the subject may be in. Suitable examples include, but are not limited to, laying, reclining, sitting, standing, walking, and combinations thereof. A posture indicator may be identified, for example, by analyzing accelerometer data for a known indicator. For example, with respect to laying, a known indicator may include the position of the subject's head in relation to the body and the like. In some embodiments, the computing device may also have a machine analysis algorithm as part of its functionality such that a library of known indicators can be updated each time the program is used.

In accordance with embodiments, a computing device can classify one or more indicators of posture and/or activity of a subject into one or more categories based on a classification rule. Further, the computing device can determine feedback for the subject based on the classification of the indicator(s) of the posture and/or activity of the subject. The classification rule can be based on one or more machine learning algorithms trained on one or more training examples. A machine learning algorithms can include one or more of the following: gradient tree boosting algorithm, a random forest algorithm, support vector machine algorithm, a penalized logistic regression algorithm, and a C5.0 algorithm.

After classification of the accelerometer data, the computing device can present feedback to the subject or another person. The computing device 102 may include one or more feedback systems for providing an output of information to the subject 104. The term “feedback” refers to any output or information provided in response to processing of the motion and/or position data. In some embodiments, feedback may be in the form of an audible or visual alert provided by the computing device 102. In some embodiments, the information or feedback information provided to the subject 104 (and/or if part of a network, the subject's caregiver, medical professional, etc.) may include time information. The time information may include any information related to a current time of day. Time information may also include a duration of time passed since the beginning of a particular activity, such as the first moment of laying down in a prone position or the start of a walk, or any other activity. In some embodiments, the activity may be determined based on analyzed motion and/or position data. In other embodiments, time information may also include additional information related to a current time and one or more other routine, periodic, or scheduled events. For example, time information may include an indication of the number of minutes remaining until the next scheduled event (e.g., scheduled washing, time to roll over to prevent bedsores, etc.), as may be determined from a calendar function or other information retrieved from a computing device 102 or server 122.

With continuing reference to FIG. 1, the system includes additional computing devices, such as the computing device 120 and the server 122, controlled by additional users (e.g., a caregiver, healthcare professional, etc.) that can communicate with the computing device 102 via the network(s) 124. Such communications among the computing device 102, the computing device 120, and the server 122 may provide additional functionality to enhance interactions of the subject 104 with his or her environment. The network(s) 124 may be a shared, public, or private network, may encompass a wide area or local area, and may be implemented through any suitable combination of wired and/or wireless communication networks. The network(s) 124 may further comprise an intranet or the Internet. In some embodiments, the sensor 106, the computing device 102, the computing device 120 of another user, and/or the server 122 may establish a connection to the network(s) 124 autonomously, for example, using a wireless module (e.g., WI-FI® communications network or a cellular communications network). Further, communication between apparatus the computing device 102, the computing device 120, and/or the server 122 may be implemented through any suitable communication channels, such as, for example, a telephone network, an extranet, an intranet, the Internet, satellite communications, off-line communications, wireless communications, transponder communications, a local area network (LAN), a wide area network (WAN), and a virtual private network (VPN).

The computing device 102 can communicate to or receive data from server 122 via the network(s) 124. For example, the computing device 120 and/or the server 122 may retrieve information from different data sources, such as the computing device's 102 database in memory 112 or the subject's social network account or other account, the Internet, and other managed or accessible databases (e.g., that of a caregiver, medical professional, etc.) stored on the server 122. It is noted that in FIG. 1, subject 104 is representative of the subject in a standing or upright position, while subject 104A is representative of the subject in a walking or active position.

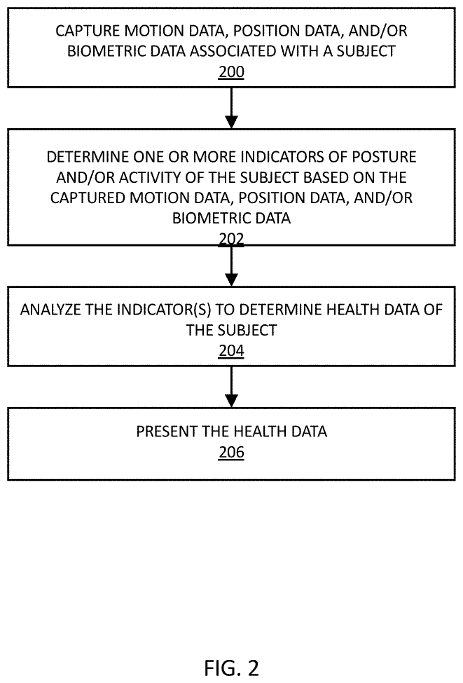

FIG. 2 illustrates a flow diagram of a method for providing health information based on motion data, position data, and/or biometric data of a subject in accordance with embodiments of the present disclosure. The method of FIG. 2 is described by example as being implemented by the system 100 shown in FIG. 1, although it should be appreciated that the method may alternatively be implemented by any other suitable system configured to acquire or capture motion data, position data, and/or biometric data of a subject.

Referring to FIG. 2, the method includes capturing 200 motion data, position data, and/or biometric data associated with a subject. For example, the sensor 106 shown in FIG. 1 may be attached to the subject 104 or otherwise mechanically connected to the subject 104 such that it can detect the motion and/or the position (e.g., location within an area and/or orientation) of the subject 104. The captured motion data, position data, and/or biometric data may be communicated to the computing device 102. In another example, the sensor 106 may be a biometric sensor configured to acquire or capture biometric data such as the subject's heartrate, heart rate variability, temperature, blood pressure, galvanic skin response, sleep quality, or the like. Further, the monitor 108 may receive the captured and store the data in memory 112. The monitor 108 may log capture motion and/or position data of the subject 104 over a period of time.

The method of FIG. 2 includes determining 202 one or more indicators of posture and/or activity of the subject based on the captured motion data, position data, and/or biometric data. Continuing the aforementioned example, the monitor 108 can determine one or more indicators of posture and/or activity of the subject 104 based on the motion data, position data, and/or biometric data stored in memory 112. An indicator, for example, can be an indicator of the subject either laying, reclining, sitting, standing, or walking. Multiple biometric data streams can be used to improve the system performance, for example, looking at heart rate in combination with accelerometry can help to distinguish periods of standing from walking in subjects who move extremely slowly.

In embodiments, the monitor 108 can determine multiple different postures and/or activities of the subject. For example, the monitor 108 can determine a posture and/or activity of the subject 104 during one period of time, and also determine a posture and/or activity of the subject 104 during a different period of time (which may be overlapping or not overlapping with the other period of time). The monitor 108 can also compare the postures and/or activities of the two periods of time. Further, the monitor 108 can determine health data of the subject 104 based on the comparison. For example, the health data can indicate whether the subject 104 performed a recommended activity, or determine whether the subject's 104 health is improving or declining.

The method of FIG. 2 includes analyzing 204 the at least one indicator to determine health data of the subject. Continuing the aforementioned example, the monitor 108 can analyze the indicator(s) to determine health data of the subject 104. The monitor 108 can determine, for the subject 104, a category among multiple, different categories based on the captured motion and/or position and/or biometric data. Further, the monitor 108 can determine feedback for the subject 104 based on the movement category. The monitor 108 can apply one or more classification schemas that are set based on one or more machine learning algorithms trained on one or more training examples. Example machine learning algorithms include, but are not limited to, a gradient tree boosting algorithm, a random forest algorithm, a support vector algorithm, a penalized logistic algorithm, and a C5.0 algorithm. Different ML algorithms can be used based on the data collected, analyzed or the types of classification outputs desired. For example, a Random Forest classifier can be trained on motion and position data to automatically discriminate between the positions using cross-validation techniques to ensure robust performance estimates.

The method of FIG. 2 includes presenting 206 the health data. Continuing the aforementioned example, the monitor 108 can use the user interface 114 to present the health data. For example, the monitor 108 can use the display 116 to present the health data. The monitor 108 may also control the user interface 114 to present, for example, a recommendation of an activity goal for the subject based on the health data. In another example, the monitor 108 may control the user interface 114 to present a prompt of an activity (e.g., walking) based on the health data. In yet another example, the monitor 108 may analyze the log of captured motion data, position data, and/or biometric data over a period of time to determine feedback for correcting activity of the subject, and also present the determined feedback via the user interface 114.

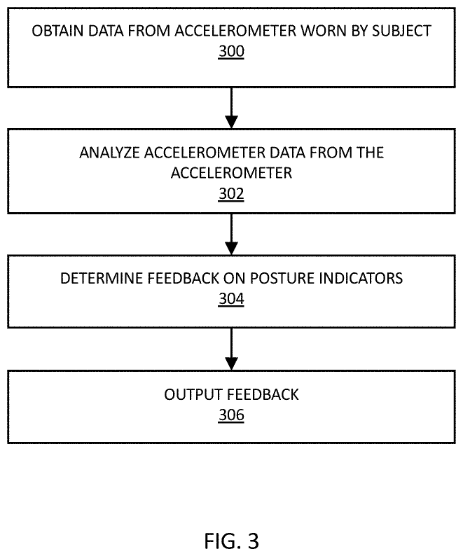

FIG. 3 illustrates a flow diagram of a method for monitoring and/or tracking a position and/or posture of a subject in accordance with embodiments of the present disclosure. The method of FIG. 3 is described by example as being implemented by the system 100 shown in FIG. 1, although it should be appreciated that the method may alternatively be implemented by any other suitable system.

Referring to FIG. 3, the method includes obtain 300 data from a sensor worn by a subject. For example, the sensor 106 shown in FIG. 1 may be an accelerometer used for capturing accelerometer data while the accelerometer is worn by the subject 104.

The method of FIG. 3, for the example of obtaining 300, includes analyzing 302 the accelerometer data to determine one or more indicators of posture of the subject. Further, the method of FIG. 3 includes determining 304 feedback on the indicator(s) of posture. For example, the monitor 108 of the computing device 102 may classify the posture information represented by the indicator(s) into one or more categories among multiple categories using a classification rule (e.g., standing, sitting, walking, laying, reclining, etc.). The monitor 108 may generate feedback in response detection of postures with associated posture information that is classified into one or more of the plurality of categories.

In some embodiments, feedback may include information associated with a category into which the posture information is classified. For example, the classification rule may be a result of training a machine learning algorithm on training examples. Examples of such machine learning algorithms include, but are not limited to, gradient tree boosting algorithm, a random forest algorithm, support vector machine algorithm, a penalized logistic regression algorithm, a C5.0 algorithm, support vector machines, Fisher's linear discriminant, nearest neighbor, k nearest neighbors, decision trees, random forests, neural networks, and combinations thereof and so forth.

In some embodiments, the feedback may include one or more recommendations for a change in the posture of the subject. For example, in response to the monitor 108 determining that a posture of the subject 104 has been held too long (e.g., laying), the monitor 108 may generate feedback to the subject 104 (and/or caregiver, medical professional, etc.) indicating the identified posture needs to be changed and/or including recommendations on how to improve and/or correct the posture. The monitor 108 may use the user interface 114 to present the feedback. By way of further example, in response to the monitor 108 determining that a posture of the subject 104 is a poor, the monitor 108 may generate feedback indicating the identification of the poor posture to the subject 104 via the user interface 114 and/or including recommendations on how to improve and/or correct the posture. For example, feedback containing an identification of a forward head posture may also include a recommendation to slide the head backwards while preserving the line of sight. In another example, feedback containing an identification of rounded shoulders posture and/or hunchback posture may also include one or more recommendations to release chest tightness, to stretch, to perform specific workout exercises that are designed to strengthen upper back muscles, and the like.

In some embodiments, the monitor 108 may determine feedback for the subject by generating one or more scores associated with one or more postures of the subject. For example, the monitor 108 may calculate one or more scores associated with the posture of the subject 104 based on indicator(s) of posture and/or based on additional posture-related information obtained by the computing device 102. Accordingly, the computing device 102 may provide feedback and/or reports to the subject 104 (and/or caregiver, medical professional, etc.) based on the one or more scores. For example, if the processing device identifies that the posture of the subject is a poor posture, or a posture held for too long of a time period, the processor may modify at least one of the one or more scores, e.g., by reducing the score(s). In some embodiments, the indicator(s) and/or additional posture-related information obtained by the computing device 102 may be aggregated. In such embodiments, one or more scores may be based on the aggregated information.

The method of FIG. 3 includes outputting 306 the feedback to the subject. Continuing the aforementioned example, the monitor 108 may control the user interface 114 to output the feedback. Example feedback includes, but is not limited to, audible feedback, visual feedback, tactile feedback (e.g., by causing vibration of the processing device), or any combination thereof. The feedback may be output to the subject 104 via a speaker included in the processing device (e.g., a smartphone, a smartwatch, a tablet, a PC, a laptop, etc.).

The method of FIG. 3 may include additional steps. For example, in some embodiments, the method of FIG. 3 may include obtaining motion information related to the motion of the subject. As used herein “motion information” may refer to information associated with the motion and/or motion pattern of the subject or with a body part of the subject, e.g., walking, running, swinging arms, or the like. Similar to the at least one posture indicator, the processing device may analyze the accelerometer data to obtain the motion information. Accordingly, step 302 of FIG. 3 may include determining at least one posture indicator and concurrently obtaining motion information. To obtain motion information, various algorithms may be used, for example, posture recognition algorithms, tracking algorithms, gesture recognition algorithms, and the like.

In accordance with embodiments, the generated feedback may also be based on motion information. Accordingly, step 304 of FIG. 3 may include determining feedback for the subject based on the indicator(s) of posture and based on obtained motion information. For example, the obtained motion information may be compared to one or more received motion records. In such an example, feedback and/or reports to the subject may be based on the comparison results. By way of further example, the obtained motion information may be classified into one or more categories using a classification rule, similar to the at least one posture indicator. In such an example, feedback may be generated if the processor obtains motion information that is classified into one or more of the plurality of categories (e.g., walking, running, etc.). Other motion information that is classified into other categories may result in no feedback being generated. Moreover, the feedback may include information associated with the category into which the motion information is classified.

In some embodiments, the monitor 108 of the computing device 102 may analyze the obtained motion information to produce one or more recommendations for changes in the motion patterns of the subject, and the feedback and/or reports may include and/or be based on the one or more recommendations. Accordingly, step 304 of FIG. 3 may include determining one or more recommendations for the subject based on the obtained motion information. For example, the monitor 108 may calculate one or more scores based on the obtained motion information, and the feedback and/or reports may include and/or be based on the one or more scores. By way of further example, the monitor 108 may aggregate the obtained motion information, and the feedback and/or reports may be based on the aggregated information.

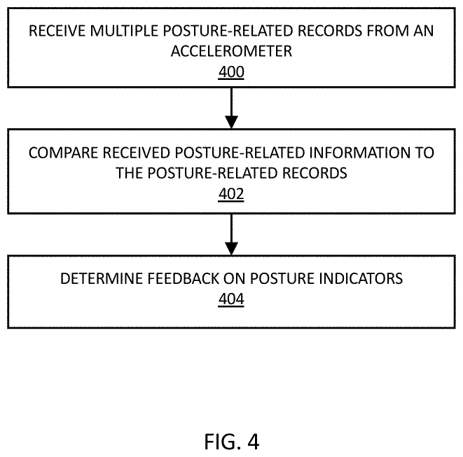

FIG. 4 illustrates a flow diagram of a method for monitoring and/or tracking a position and/or posture of a subject in accordance with embodiments of the present disclosure. The method of FIG. 4 is described by example as being implemented by the system 100 shown in FIG. 1, although it should be appreciated that the method may alternatively be implemented by any other suitable system.

Referring to FIG. 4, the method includes receiving 400 multiple posture-related records from accelerometer (e.g., accelerometer data). For example, the monitor 108 of the computing device 102 shown in FIG. 1 may analyze accelerometer data captured by accelerometer (e.g., sensor 106). In embodiments, each posture-related record may be calculated based on various aspects of the accelerometer data. For example, the accelerometer data may have been captured at different times. The posture-related records may be stored in memory 112 and retrieved by the monitor 108.

The method of FIG. 4 includes comparing 402 posture-related information to the posture-related records. For example, the monitor 108 may analyze accelerometer data captured by the accelerometer in order to obtain posture-related information. In some embodiments, the posture-related information may be based on different accelerometer data than the posture-related records. For example, the posture-related information may be based on currently captured accelerometer data while the posture-related records may be based on stored (e.g., historical) accelerometer data.

In some embodiments, the monitor 108 may compare the posture-related information to the posture-related records using a similarity function. A similarity function may accept the posture-related information and one or more posture-related records as input and then output a similarly score. For example, the posture-related information and one or more posture-related records may be embedded within a mathematic space before being input to the similarity function. In one example, each posture-related record may include information related to previously-observed posture of the subject. In such an example, the monitor 108 may use the comparison results to calculate and/or update statistics about the subject's posture over time. The feedback and/or reports may then include information derived from the statistics.

In another example, each posture-related record may include information related to an “example” posture. In such an example, the comparison results may indicate a closest or most similar example posture to the observed posture, as represented by the posture-related information. The feedback and/or reports may subsequently be based on the identity of the closest or most similar example posture. Furthermore, the monitor 108 may calculate and/or update statistics based on the identity of the closest or most similar example posture, and the feedback and/or reports may include information derived from the statistics.

In yet another example, the posture-related records may include prototype examples produced by applying statistical and/or machine learning algorithms, such as principal postures produced by principal component analysis, centers of cluster produced by data clustering, and so forth, on instances of information related to example postures. In such an example, the comparison results may indicate the closest or most similar prototype to the observed posture. The feedback and/or records may then be based on the identity of the closest or most similar prototype. Furthermore, the monitor 108 may calculate and/or update statistics based on the identity of the closest or most similar prototype, and the feedback and/or reports may include information derived from the statistics.

The method of FIG. 4 includes determining 404 feedback for the subject based on the comparison. For example, the monitor 108 may determine feedback as described herein. In addition, the feedback and/or reports to the subject (or subject's caregiver, medical professional, etc.) may be based on the comparison results.

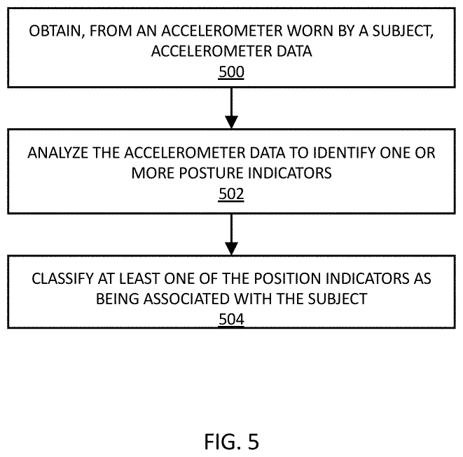

FIG. 5 illustrates a flow diagram of a method for monitoring and/or tracking a position and/or posture of a subject in accordance with embodiments of the present disclosure. The method of FIG. 5 is described by example as being implemented by the system 100 shown in FIG. 1, although it should be appreciated that the method may alternatively be implemented by any other suitable system.

Referring to FIG. 5, the method includes obtaining 500, from an accelerometer worn by a subject, accelerometer data. For example, the sensor 106 shown in FIG. 1 can be used to capture accelerometer data of the subject 104. The accelerometer data may be received by the monitor 108.

The method of FIG. 5 includes analyzing 502 the accelerometer data to identify one or more posture indicators. Continuing the aforementioned example, the monitor 108 may identify positions/postures such as laying, reclining, sitting, standing, walking, and the like. The monitor 108 may employ a suitable algorithm to identify body parts and may integrate one or more machine learning techniques with such algorithm(s).

The method of FIG. 5 includes classifying 504 at least one of the position indicators as being associated with the subject. Continuing the aforementioned example, various accelerometer data aspects and features may be considered for the determination, including identifying the position/posture of the subject, the amount of time spent in said position/posture, etc. The monitor 108 may also make the determination based on one or more other criteria, including, for example, a confirmation or rejection signal from the subject, or other external information such as an auditory signal, as well as historical information regarding an environmental situation or other learned and/or programmed assessments of the situation.

In some embodiments, the classification of the at least one indicator of the posture of the subject into at least one of a plurality of categories based on a classification rule, and the feedback for the subject is determined based on the classification of the at least one indicator of the posture of the subject. In one embodiment, the classification rule is based on one or more machine learning algorithms trained on one or more training examples. In some embodiments, the one or more machine learning algorithms correspond to one or more of the following: gradient tree boosting algorithm, a random forest algorithm, support vector machine algorithm, a penalized logistic regression algorithm, a C5.0 algorithm, and combinations thereof. Further, the analyzed and classified data are compiled and outputted to the subject (and/or the subject's caregiver, medical professional, etc.) as described herein.

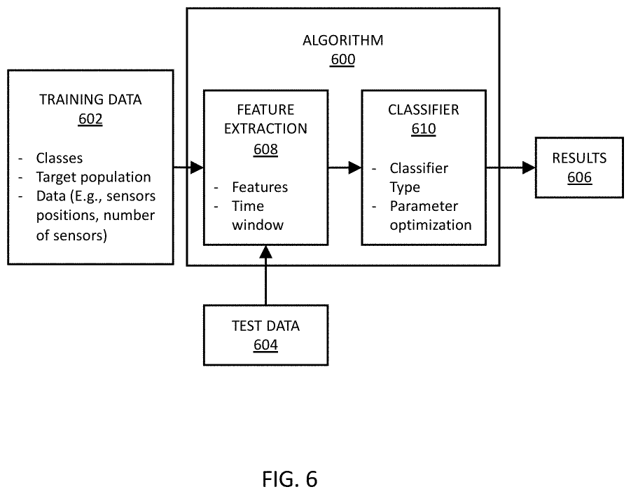

In other embodiments, the methods further comprise customizing the classifier to enable a user, such as a caregiver, medical professional, etc., to specifically train the customized classifier on a specific population/sensor placement. This can be beneficial, for example, because many accelerometers and algorithms are trained on a specific population and specific sensor placement which leaves little room for customization between research studies or clinical use cases In such examples, and as shown in FIG. 6, the method can alter one or more parameters of the test data that is fed into the algorithm thereby getting results specific to the desired population/sensor placement. Such methods provide that once the algorithm design parameters have been selected (e.g. classifier type, features), these can remain the same regardless of the specific classification problem.

FIG. 6 illustrates a diagram depicting application of an example algorithm 600 to training data 602 and test data 604 to generate results 606 in accordance with embodiments of the present disclosure. Referring to FIG. 6, training data 602 includes, but is not limited to classes, target population, and data (e.g., sensor positions data and number of sensors) of which the ground truth (e.g. labeling) is known. Training data can be used to teach the classifier the patterns of interest. Test data 604 is a sub-set of the labeled training data that is set aside and used to see how well the machine can predict based on its training. The algorithm 600 may apply a feature extraction function 608 to the training data 602 to generate features used to train the classifier. The algorithm 600 includes a classifier 610 to generate a classifier type and parameter optimization based on the input from the feature extraction function 608.

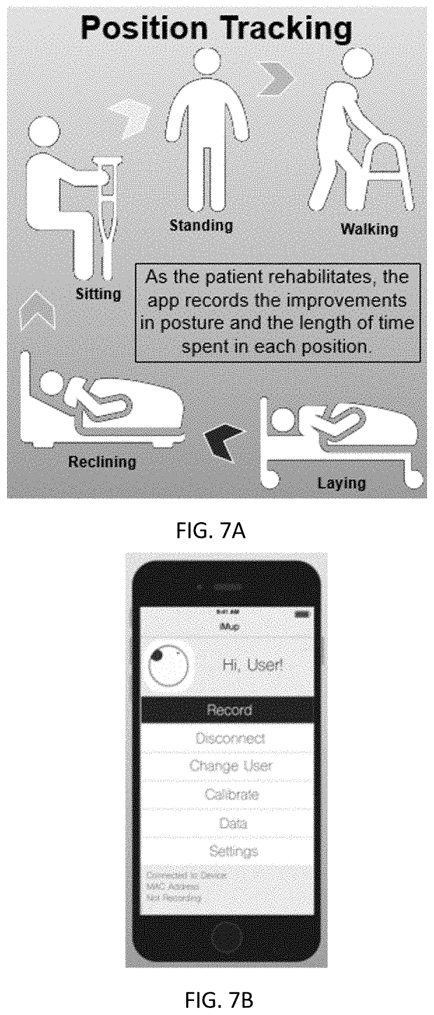

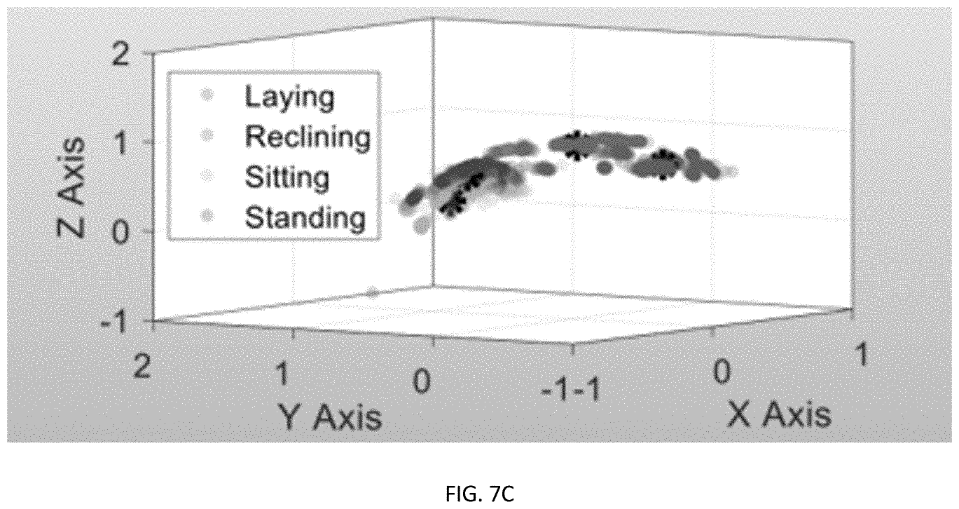

In an example use scenario, FIGS. 7A-7C illustrate diagrams and a smartphone screen display for providing health information based on motion and/or position data of a subject in accordance with embodiments of the present disclosure. In this example, the system can be used for the goal of shortening hospital stay of a patient by posture improvement. It is noted that any angle increase of the patient may be considered an improvement of the patient's condition. The system may include a smartphone having an application residing thereon and being communicatively connected to a motion sensor. Referring to FIG. 7A, the diagram shows the progression of states of the patient beginning with the state of laying to the state of walking. The progression includes the states of reclining, sitting, and standing. As the patient rehabilitates, the application may record the improvements in posture and the length of time spent in each position.

FIG. 7B shows an example screen display of the smartphone. This screen display show buttons that the user (e.g., patient) can interact with to initiate disconnecting, changing a user, calibrating, viewing data, and managing settings. “Record” can be selected to start an open recording (e.g., data is now being streamed from sensor). “Disconnect” can be selected prior to disconnecting present sensor. “Change User” can be selected such that a person can enter another username. “Calibrate” can be selected to collect the initial training data by going through the different postures. “Data” can be selected to allow a user to manually send data (in a full version, this can happen automatically/in real-time).

FIG. 7C is a graph showing results of initial trails. In this example, the sensor was worn on the patient's shoulder. Referring to FIG. 7C, the figure is showing positioning arc for sensor worn on shoulder. Particularly, the graph is showing that as someone is moving from laying to standing their shoulder is moving in an expected pattern.

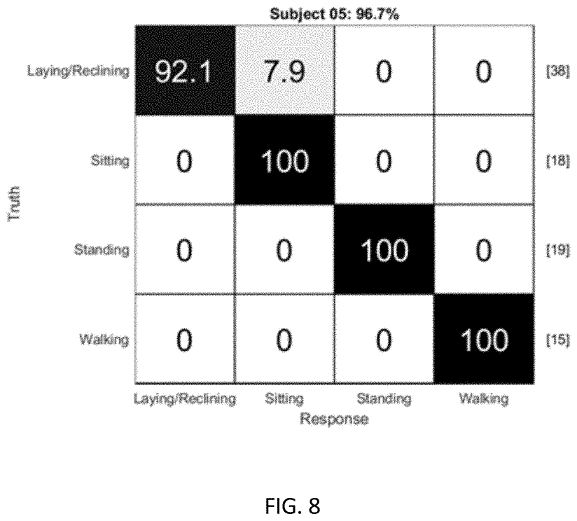

FIG. 8 illustrates a confusion matrix of data for hospitalized adult. For this, laying/reclining was combined into one group because clinical implication between the two groups is not high. This matrix shows a 92.1% accuracy for laying/reclining (number of times the algorithm was able to guess correctly), and 100% for rest.

In accordance with embodiments, a system disclosed herein can measure activity and position of people while recovering in a hospital. The system can accept data from a variety of sensors including, but not limited to, accelerometer, gyroscope, heart rate, GPS, beacon, and the like. Further, the system can automatically recognize and classify the patient's activity and position based on machine trained algorithms based on population wide performance data. The system can also automatically recognize and classify the patient's activity and position based on machine trained algorithms custom built from the patient's own performance data. Further, the system can generate reports for medical and care providers summarizing these data for use in care planning. The system can also automatically create activity goals for the next time period. Further, the system can provide patient cues and reminders to increase or limit activity as appropriate. The system can also provide care provider cues to provide opportunities to assist the patient increase activity.

In accordance with embodiments, two classification tasks have been provided. These classification tasks include resting (laying, reclining, and sitting) vs. activity (standing and walking) and prone (laying and reclining) vs. upright (sitting, standing, and walking) to demonstrate that sensor position is important based on the task and switching sensor position without retraining the algorithm does not produce accurate results. For resting vs. activity, the accelerometer worn on the leg can provide better classification performance than using the chest sensor position. This is indicated in confusion matrices, where a higher percent value for the diagonal starting in the upper left corner of each matrix shows a better performance for the classifier while higher percentages off the diagonal shows poor classifier performance. Additionally, in the classification task prone vs. upright, the chest position provided better classification than the leg sensor position for this task, showing a 97.8% and 99.7% response accuracy for prone and upright tasks, respectively for the chest sensor. Using a sensor on the leg when the classifier had been trained with a chest positioned sensor decreased the accuracy of the task. For these tasks, changing the sensor to the chest when the classifier had been trained on the leg did not have a negative impact.

In accordance with embodiments, a system as disclosed herein can relay movement data in a “shift-change” report that details time spent in each position as well as other metrics to support acknowledging the wearer's movements throughout the specified time period. This data can connect with the medical health record to ensure proper tracking and monitoring by all of care team. Data can also be shown to clinical staff on an ‘as needed’ basis, in other words, the clinical staff can pull reports in real time to get more frequent updates as needed. Patients can also have access to their data to support individual changes to movement throughout a time period. This data can be relayed as a summary report.

The present subject matter may be a system, a method, and/or a computer program product. The computer program product may include a computer readable storage medium (or media) having computer readable program instructions thereon for causing a processor to carry out aspects of the present subject matter.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a RAM, a ROM, an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network, or Near Field Communication. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++, Javascript or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present subject matter.

Aspects of the present subject matter are described herein with reference to flowchart illustrations and/or block diagrams of methods, apparatus (systems), and computer program products according to embodiments of the subject matter. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present subject matter. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the embodiments have been described in connection with the various embodiments of the various figures, it is to be understood that other similar embodiments may be used, or modifications and additions may be made to the described embodiment for performing the same function without deviating therefrom. Therefore, the disclosed embodiments should not be limited to any single embodiment, but rather should be construed in breadth and scope in accordance with the appended claims.

What is claimed is:

1. A system comprising:
at least one sensor configured to capture motion data, position data, and/or biometric data associated with a subject; and
a monitor configured to:
determine at least one indicator of one of posture and activity of the subject based on the captured motion data, position data, and/or biometric data;
analyze the at least one indicator to determine health data of the subject; and
present the health data.

2. The system of claim 1, wherein the at least one indicator comprises one of an indicator of laying, an indicator of reclining, an indicator of sitting, an indicator of standing, and indicator of walking, activity or inactivity, and sleep quality.

3. The system of claim 1, wherein the at least one sensor comprises an accelerometer, a gyroscope, a heart monitor, a beacon component, a position sensor.

4. The system of claim 1, wherein the monitor comprises one of a computing device, a smartphone, a tablet computer, and a smartwatch.

5. The system of claim 1, wherein the at least sensor is configured to be attached to one of the subject's body, clothing, head, torso, arms and/or legs while capturing the motion and/or position and/or other biometric data .

6. The system of claim 1, wherein the monitor is configured to:
log a plurality of captured motion data, position data, and/or biometric data associated with the subject over a period of time;
analyze the log of the plurality of captured motion data, position data, and/or biometric data to determine feedback for correcting activity of the subject; and
present the determined feedback.

7. The system of claim 1, wherein the monitor is configured to:
determine, for the subject, a movement category among a plurality of movement categories based on the captured motion data, position data, and/or biometric data;
determine feedback for the subject based on the movement category; and
present the determined feedback.

8. The system of claim 7, wherein the monitor is configured to determine the movement category by determining the type of output/feedback desired.

9. The system of claim 8, wherein one or more classification rules are set based on one or more machine learning algorithms trained on one or more training examples and existing training data sets from the subject and/or other subjects.

10. The system of claim 9, wherein training data is collected and labeled for use in machine learning algorithms.

11. The system of claim 8, wherein one or more classification rules are set based on one or more machine learning algorithms trained on one or more training examples.

12. The system of claim 8, wherein the monitor is configured to use a predetermined machine learning tool and technique for training a classifier to make decisions based on labeled training data.

13. The system of claim 1, wherein the monitor is configured to:
determine a first posture and/or activity of the subject based on motion data, position data, and/or biometric data captured at a first time period;
determine a second posture and/or activity of the subject based on motion data, position data, and/or biometric data captured at a second time period, wherein the second time period occurs subsequent to the first time period;
compare the first posture and/or activity with the second posture and/or activity; and
determine the health data based on the comparison.

14. The system of claim 1, further comprising a user interface configured to present the health data.

15. The system of claim 14, wherein the user interface comprises a display, and wherein the monitor is configured to control the display to display the health data.

16. The system of claim 1, wherein the monitor is configured to present a recommendation of an activity goal for the subject based on the health data.

17. The system of claim 1, wherein the monitor is configured to present a prompt of an activity for the subject based on the health data.

18. The system of claim 1 further comprising a recorder configured to facilitate the collection of labeled training data.

19. A method comprising:
capturing motion data, position data, and/or biometric data associated with a subject;
determining at least one indicator of one of posture and activity of the subject based on the captured motion data, position data, and/or biometric data;
analyzing the at least one indicator to determine health data of the subject; and
presenting the health data.

20. The method of claim 19, wherein the at least one indicator comprises one of an indicator of laying, an indicator of reclining, an indicator of sitting, an indicator of standing, and indicator of walking, activity or inactivity, and/or sleep quality.

21. The method of claim 19, wherein the at least one sensor comprises one of an accelerometer, a gyroscope, a heart monitor, a beacon component, and a position sensor.

22. The method of claim 19, further comprising:
determining, for the subject, a movement category among a plurality of movement categories based on the captured motion data, position data, and/or biometric data;
determining feedback for the subject based on the movement category; and
presenting the determined feedback.

23. The method of claim 22, further comprising determining the movement category by applying one or more classification algorithms.
